(19) United States
(12) Patent Application Publication
(10) Pub. No.: US 2024/0050634 A1
(43) Pub. Date: Feb. 15, 2024

(54) Bypass Line, Medical Treatment Apparatus, and Method

(71) Applicant: Fresenius Medical Care Deutschland GmbH, Bad Homburg (DE)

(72) Inventor: Juergen Haecker, Bad Homburg (DE)

(21) Appl. No.: 18/266,965

(22) PCT Filed: Dec. 10, 2021
(86) PCT No.: PCT/EP2021/085232

§ 371 Date: Jun. 13, 2023

(30) Foreign Application Priority Data
Dec. 14, 2020 (DE) 102020133387.0

(51) Int. Cl. A61M 1/16; A61M 39/10

(52) U.S. Cl. CPC A61M 1/165; A61M 39/10

(57) ABSTRACT

The present disclosure relates to a bypass line for establishing a fluid communication between a dialysis liquid inlet line of a treatment apparatus, by which dialysis liquid is fed out of the interior of the treatment apparatus and into an exterior of the treatment apparatus, and a dialysate outlet line, by which dialysate is fed back from the exterior into the interior of the treatment apparatus. The bypass line has at least a first connector, a second connector and a non-return valve. The first connector is used to connect the bypass line to a connector of the dialysis liquid inlet line to achieve a fluid communication. The second connector is for connecting the bypass line to a connector of the dialysate outlet line while achieving a fluid communication. The non-return valve only allows flow through the bypass line from the first connector towards the second connector.

### CROSS-REFERENCE TO RELATED APPLICATIONS

The present application is the national stage entry of International Application No. PCT/EP2021/085232, filed on Dec. 10, 2021, and claims priority to Application No. DE 102020133387.0, filed in the Federal Republic of Germany on Dec. 14, 2020, the disclosures of which are expressly incorporated herein in their entirety by reference thereto.

### TECHNICAL FIELD

The present disclosure relates to a bypass line as described herein. The present disclosure also relates to methods as described herein, a control device as described herein, and a medical treatment apparatus as described herein.

### BACKGROUND

In practice, medical treatment apparatuses which guide fluid via a first line to an external, exchangeable component, such as a blood filter or a dialyzer, and guide fluid out of this component via a second line, regularly have a bypass line.

The latter serves to connect the first line to the second line without said component being integrated into the bypass fluid paths created, for example, for flushing the system, the two lines, etc.

An aspect of the present disclosure is to specify a further bypass line suitable for this purpose. Furthermore, a further method for checking a condition of the bypass line, a further control device and a further medical treatment apparatus are to be specified.

### SUMMARY

The aspect according to the present disclosure is achieved with a bypass line as described herein. It is further achieved by a method as described herein, a control device as described herein, and a medical treatment apparatus as described herein.

According to the present disclosure, a bypass line for establishing a fluid communication between at least one first line, e.g., a dialysis liquid inlet line, and a second line, e.g., a dialysate outlet line of a treatment apparatus, e.g., a blood treatment apparatus, in particular a dialysis apparatus, is proposed.

When used as intended, dialysis liquid is passed out of the interior of the treatment apparatus into an exterior of the treatment apparatus via the dialysis liquid inlet line.

Using the dialysate outlet line, dialysate is returned from the exterior into the interior of the treatment apparatus when used as intended.

The bypass line described herein has a first connector. The first connector is used to connect the bypass line to a first connector of the dialysis liquid inlet line, whereby a fluid communication between the bypass line and the dialysis liquid inlet line is achieved.

The bypass line also has a second connector. The second connector is used to connect the bypass line to a second connector of the dialysate outlet line, again with the aim of achieving fluid communication.

Additionally, the bypass line includes at least one non-return valve, particularly a check-valve, which allows flow through the bypass line only or essentially only, from the first connector towards the second connector.

The previously referred to first and second connectors are, in certain embodiments, the only connectors of the bypass line.

According to the present disclosure, a method for checking a condition of the bypass line described herein is proposed, which is connected to a dialysis liquid inlet line and dialysate outlet line of a provided medical treatment apparatus. The medical treatment apparatus includes the dialysis liquid inlet line with a connector which is provided for connecting the dialysis liquid inlet line to a blood filter of the medical treatment apparatus. Further, the medical treatment apparatus includes the dialysate outlet line with a connector which is provided for connecting the dialysate outlet line to the blood filter. In addition, the medical treatment apparatus has a conveying device for conveying fluid within the dialysis liquid inlet line and/or within the dialysate outlet line.

The method is to be carried out when or after the bypass line has been connected by its first connector to the connector of the dialysis liquid inlet line and by its second connector to the connector of the dialysate outlet line.

The method described herein includes actuating the conveying device, in particular in an attempt or with the aim of conveying fluid within or along the dialysis liquid inlet line and/or within or along the dialysate outlet line, in order to build up pressure or negative pressure within the dialysis liquid inlet line and/or within the dialysate outlet line.

The method further includes, at and/or for a first point in time, measuring or otherwise determining the pressure prevailing within the dialysis liquid inlet line and/or within the dialysate outlet line. This pressure is also referred to herein as the initial pressure.

The method further includes determining at least one pressure value based on the measured pressure, for example as an indication of a numerical value with the dimension mbar, hPa, mmHg or the like, in the form of an absolute value, a relative value, a difference, and so forth.

The method further includes evaluating the at least one pressure value, for example according to predetermined criteria.

The method further includes emitting or outputting a signal which indicates the condition of the bypass line and is a result of the evaluation.

According to the present disclosure, a control device is also proposed which is programmed to carry out a test or method for checking the condition of a bypass line, preferably a bypass line as described herein, provided that or wherein the bypass line is connected via the aforementioned connectors to both the dialysis liquid inlet line and the dialysate outlet line of a medical blood treatment apparatus. The test includes the steps of the method described herein in any embodiment.

According to the present disclosure, a medical treatment apparatus is further proposed which includes a housing, which can separate an interior and an exterior of the treatment apparatus, and optionally a bypass line as described herein.

Embodiments according to the present disclosure may include some, any or all of the following features in any combination, as long as it is not recognized to be technically impossible by the person skilled in the art.

In all of the previous and following statements, the use of the expression “may be” or “may have” and so on, is to be understood synonymously with “preferably is” or “preferably has,” and so on respectively, and is intended to illustrate embodiments according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” as encompassing “at least one”. This understanding is also equally encompassed by the present invention as the interpretation that a numeric word, for example, “one” may alternatively mean “exactly one”, wherever this is evidently technically possible for the person skilled in the art. Both understandings are encompassed by the present disclosure and apply herein to all used numerical words.

Whenever spatial references such as “above”, “below”, “upper” or “lower” are mentioned herein, when in doubt the person skilled in the art understands these as a spatial indication with reference to the orientation in the attached figures and/or the arrangement of the bypass line according to the present disclosure in its intended use.

Whenever an embodiment is mentioned herein, this always represents an exemplary embodiment according to the present disclosure, which is not to be understood as limiting.

Embodiments disclosed herein can be in any combination with each other as desired, the present disclosure also encompasses this.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g., formulated as negation, is also disclosed.

Whenever a suitability or a method is mentioned herein, the present disclosure also includes a corresponding programming or configuration of a device or a section thereof suitable and/or programmed for this or for carrying out the method.

Whenever a time point is referred to herein, this may alternatively be a period of time.

In some embodiments of the method described herein, the result of the evaluation is or includes a faulty connection of the by-pass line to the dialysis liquid inlet line and/or the dialysate outlet line or is or includes an indication of this.

Additionally or alternatively, the evaluation of the at least one pressure value is or encompasses comparing it with a, for example first, reference value or reference range.

In each of the embodiments described herein, a faulty connection can be concluded as a result, or it is concluded if the evaluation of the pressure value, the evaluation of a difference from several pressure values and/or the comparison of pressure values that were determined for different lines results, that a reference value established or predetermined for this purpose is exceeded or the pressure value lies outside a reference range established or predetermined for this purpose.

Reference values and/or reference ranges can be designed in certain embodiments in such a way that a faulty connection can be concluded as a result if the evaluation, as stated above, shows that the value falls below a specified or predetermined reference value or the pressure value is within a specified or predetermined reference range.

In several embodiments, a fault free connection may be concluded as a result, or is concluded when the evaluation of the pressure value indicates that it does not exceed a reference value, e.g., the first reference value, or is within, for example, the first reference range, or vice versa.

Reference values or reference ranges may be or encompass limit values or be limited by such.

In several embodiments, the result is or encompasses a malfunction of the non-return valve of the bypass line or is or encompasses an indication thereof.

Alternatively or additionally, the result is or encompasses that there is a faulty connection. This may be due to, be a consequence of, or require a connection of the connector of the dialysis liquid inlet line to the second connector of the bypass line, which should be connected to the dialysate outlet line, and a connection of the connector of the dialysate outlet line to the first connector of the bypass line, which should be connected to the dialysis liquid inlet line.

In some embodiments of the method described herein, to determine the pressure value, both the pressure prevailing in the dialysis liquid inlet line at the first time point and the pressure prevailing in the dialysate outlet line at the first time point are or have been measured. In these, or also in other, embodiments, a pressure difference is determined preferably between the pressure measured or the pressure value determined in the dialysis liquid inlet line and the pressure measured or the pressure value determined in the dialysate outlet line or vice versa.

The evaluation of the at least one pressure value is or encompasses in these embodiments a comparison of this pressure difference, for example, with a second reference value or reference range therefor.

In certain embodiments, a faulty connection can be concluded as a result if the evaluation of the pressure value shows that the pressure value exceeds the second reference value or lies outside the second reference range.

In several embodiments, the method described herein encompasses as a further step measuring a pressure prevailing in the dialysis liquid inlet line and/or in the dialysate outlet line at a second time point, which is after the first time point. Additionally, it encompasses determining at least a second pressure value based thereon.

In these embodiments, evaluating the pressure value is or encompasses a comparison of the pressure difference between the pressure measured in the dialysis liquid inlet line at the first and second time points or the pressure values determined based on these and/or a comparison of the pressure difference between the pressure measured in the dialysate outlet line at the first and second time points or the pressure values determined based on these, i.e., by preferably taking the initial pressures into account, with a, for example, third or fourth reference value or reference range therefor.

In certain embodiments, a faulty connection may be concluded as a result if the evaluation of the pressure values is or encompasses that at least one of them exceeds the third or fourth reference value or is outside the third or fourth reference range, respectively.

In some embodiments, the conveying device is actuated with the aim of generating a negative pressure in both the dialysis liquid inlet line as well as in the dialysate outlet line. Additionally, the method described herein encompasses releasing the negative pressure generated in the dialysate outlet line.

In these embodiments, measuring a pressure prevailing in the dialysis liquid inlet line and/or in the dialysate outlet line encompasses a measurement at one or more time points after the pressure release.

In several embodiments, actuating the at least one conveying device and/or the building of pressure or negative pressure takes place via an open first valve which is arranged upstream of the connector of the dialysis liquid inlet line but downstream of the first pressure sensor. Alternatively or additionally, the actuating or building of pressure or negative pressure takes place via an open second valve which is arranged downstream of the second connector of the dialysate outlet line but upstream of the second pressure sensor.

In some embodiments, the release of the prevailing negative pressure in the dialysate outlet line is achieved by connecting the dialysate outlet line to a fresh water source or to a drain or encompasses this connection.

In some embodiments, the negative pressure is released by opening a valve to a fresh water line or a valve to a drain or outlet line or includes this opening.

In some embodiments, the emitted or output signal is associated with or is coded for a faulty condition of the bypass line.

This may particularly be the case if evaluating the pressure value results in or encompasses its exceeding a reference value or leaving a reference range. Alternatively or additionally, if determining the pressure value based on measured pressures which were measured within the dialysis liquid inlet line, after the pressure was released results in a pressure increase.

In some embodiments, the method described herein further encompasses fluidically shutting off the dialysate outlet line, or a section of it, after releasing the negative pressure in the dialysate outlet line in such a way that there is no longer any connection between the dialysate outlet line and the fresh water source or the drain.

In these embodiments, in order to determine the at least one pressure value at least two pressures are measured in the dialysate outlet line at two different time points after it has been blocked.

In addition, in these embodiments a signal is emitted or output which is associated with a faulty condition of the bypass line, in particular if evaluating the at least one pressure value detects a pressure change within the dialysate outlet line as a result.

In several embodiments of the medical treatment apparatus described herein, the bypass line can be completely or at least partially accommodated in the interior or in the housing of the treatment apparatus. Hereby, the first connector and the second connector of the bypass line are preferably accessible to the user from an exterior of the treatment apparatus, in order to be connected as herein described.

In some embodiments, the medical treatment apparatus includes a control device as described herein.

Alternatively or additionally, the medical treatment apparatus includes a dialysis liquid inlet line with a connector provided for its connection to a blood filter, a dialysate outlet line with a connector provided for its connection to a blood filter, and a conveying device for conveying fluid within the dialysis liquid inlet line and/or within the dialysate outlet line.

A condition of the bypass line described herein can be understood as, for example, the presence of the bypass line, the intactness of the bypass line, the correct arrangement of the bypass line on the medical treatment apparatus, the correct connection of the bypass line with the dialysis liquid inlet line and/or with the dialysate outlet line and/or the functionality of the bypass line, in particular the functionality of the non-return valve arranged within the bypass line.

In several embodiments, the evaluation of at least one pressure value is or encompasses determining a change in the pressure, for example by comparison with limit values, threshold values, value ranges and/or is or includes determining the duration until a change occurs, etc.

Furthermore, in certain embodiments, the method includes evaluating the determined change based on predetermined criteria or variables such as limit values, threshold values, value ranges and/or the duration until the, or a, predetermined pressure change has occurred etc.

The predetermined criteria and quantities can be stored, for example, in a storage device, for example the medical treatment apparatus, and be read from there.

In some embodiments, the method described herein can include, as a further step, an optional output of the signal or a corresponding signal to the user of the medical treatment apparatus. The signal to the user, e.g., visual, acoustic or the like, can inform the user that the evaluation, e.g., the determined change, satisfies or does not satisfy predetermined conditions.

In some embodiments, the pressure measurements, which take place at the first time point and/or at the second time point, and/or with which the initial pressure or pressure prevailing within the dialysis liquid inlet line and/or within the dialysate outlet line is measured, is carried out via—or using—a first pressure sensor arranged upstream of the first connector in or on the dialysis liquid inlet line and/or via a second pressure sensor arranged downstream of the second connector in or on the dialysate outlet line.

In some embodiments, the at least one conveying device is an ultrafiltration pump and/or a second flow pump arranged downstream of the dialysate outlet line.

In some embodiments, actuating the delivery device encompasses establishing a negative pressure or pressure both upstream of the dialysis liquid inlet line connector and downstream of the dialysate outlet line connector.

In some embodiments, actuating the at least one conveying device at the first time point may be performed such that substantially equal pressure values are preferably present at the first pressure sensor and the second pressure sensor.

In several embodiments of the medical treatment apparatus described herein, a connection to the atmosphere may be established between the first time point and the second time point downstream of the second connector and/or to the dialysate outlet line.

In certain embodiments of the present disclosure, the fluid conveyed is a dialysis fluid, and in some embodiments according to the present disclosure, the fluid conveyed is a dialysis liquid produced and/or provided on-line by a treatment apparatus.

The treatment apparatus described herein, is in certain embodiments designed as an extra corporeal treatment apparatus in particular as an extra corporeal blood treatment apparatus, such as a dialysis apparatus, in particular a hemodialysis apparatus, hemofiltration apparatus or hemodiafiltration apparatus and so forth.

The present disclosure further relates to a digital particularly non-volatile storage medium, particularly in the form of a machine readable carrier, particularly in the form of a diskette, memory card, CD, DVD EPROM, FRAM (Ferroelectric RAM) or SSD (Solid-State-Drive), particularly with electronically or optically readable control signals. This can interact with a programmable computer system so that a control device will be or is configured to be a control device according to the present disclosure, via which the method according to the present disclosure may be initiated. Alternatively, the storage medium may be configured to interact in such a way with a conventional medical treatment apparatus that this is reprogrammed to be a medical treatment apparatus according to the present disclosure.

The present disclosure further relates to a computer program product, which includes a volatile, transient or stored on a machine-readable carrier program code or a signal wave, which are suitable for configuring a control device into a control device described herein via which the method described herein may be initiated when the computer program runs on a computer. Alternatively, the computer program-product may be configured to interact in such a way with a conventional medical treatment apparatus that this is reprogrammed to be a medical treatment apparatus described herein.

Computer program product, for example, can be understood according to the present disclosure as a computer program stored on a carrier, an embedded system being a comprehensive system with a computer program (e.g., electronic device with a computer program), a network of computer implemented computer programs (e.g., client/server-system, a cloud computing system, etc.), or a computer on which a computer program is loaded, runs, is stored, is executed or developed.

The term “machine readable carrier” as is as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage or storage medium referred to herein.

The present disclosure also relates to a computer program, which includes a program code via which a conventional control device may be configured into a control device described herein via which the method described herein may be initiated. Alternatively, the computer program is suitable to interact in such a way with a conventional medical treatment apparatus that this is reprogrammed to be a medical treatment apparatus as described herein.

Some or all embodiments according to the present disclosure may include one, some or all of the advantages mentioned above or in the following.

An advantage may be to reduce or totally eliminate the risk of cross-contaminations from one side, e.g., the dialysate side, to the other side, e.g., the hydraulic side, of the treatment apparatus. Since in using the non-return valve a swapping of the connectors of the bypass line can be detected by monitoring the pressure profile, in case of swapped connectors the need for disinfection before the next treatment can be indicated.

The functionality of the by-pass line can be further advantageously checked and a corresponding indication can be communicated to the user.

All advantages achievable with the method described herein can also be achieved undiminished with the devices described herein, and vice versa.

### BRIEF DESCRIPTION OF THE DRAWINGS

The present disclosure is described below with reference to the accompanying figures. In the drawing, identical reference numerals denote identical or similar elements. The following applies:

FIG. 1 shows in a simplified schematic a fluid line diagram of a medical treatment apparatus.

FIG. 2 shows in a simplified schematic a section of the fluid line diagram of FIG. 1 having a bypass line.

FIG. 3 shows in a simplified schematic the course of the method described herein in an embodiment.

### DETAILED DESCRIPTION

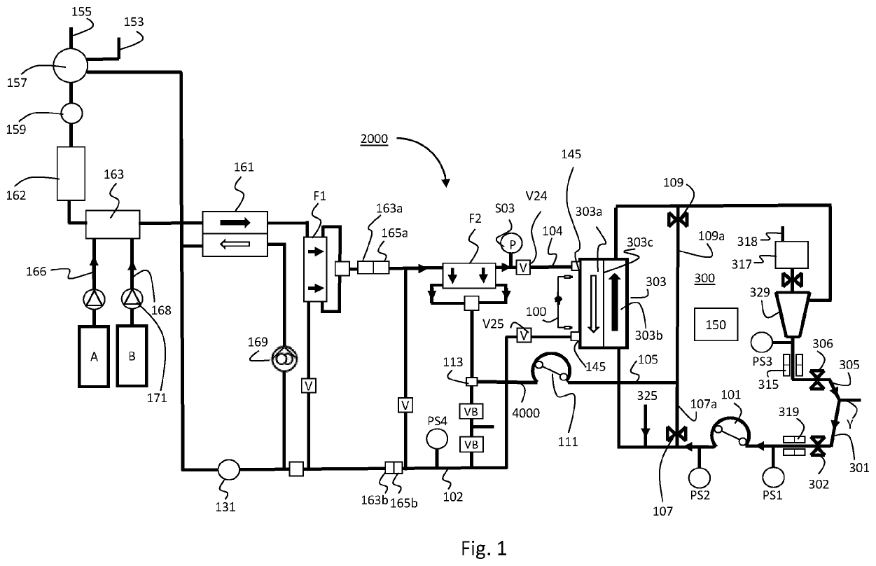

FIG. 1 shows a process flow chart of a medical treatment apparatus 2000, in this case a blood treatment apparatus, connected to an extracorporeal blood circuit 300, which can be connected to the vascular system of the patient (not shown) via a double-needle access for treatment, or via a single-needle access using for example, an additional Y-connector (reference numeral Y) as shown in FIG. 1. The blood circuit 300 may optionally be in sections in or on a blood cassette.

Pumps, actuators and/or valves in the area surrounding the blood circuit 300 are connected to the treatment apparatus 2000 or, for example, to a control device 150 encompassed by the treatment apparatus 2000.

The blood circuit 300 includes (or is connected to) an arterial patient tubing clamp 302 and an arterial connection needle of an arterial section or an arterial patient line, blood withdrawal line, or first line 301. The blood circuit 300 further includes (or is connected to) a venous patient tubing clamp 306 and a venous connection needle of a venous section, venous patient line, blood return line, or second line 305.

A blood pump 101 is provided in or on the first line 301, a substitute pump 111 is connected to a dialysis liquid inlet line 104 for conveying fresh dialysis liquid which is filtered in a further filter stage (F2) (substituate). A substituate line 105 can be fluidically connected to the inlet line 104. Using the substituate pump 111, substituate can be introduced by pre-dilution, via a pre-dilution valve 107, or by post-dilution, via a post-dilution valve 109, via associated lines 107a or 109a into line sections, for example into the arterial line section 301 or into the venous line section 305 (here between a blood chamber 303b of a blood filter 303 and a venous air separation chamber or venous blood chamber 329) of the blood circuit 300.

The blood filter 303 includes the blood chamber 303b connected to the arterial line section 301 and to the venous line section 305. A dialysis fluid chamber 303a of the blood filter 303 is connected to the dialysis liquid inlet line (104) leading to the dialysis liquid chamber 303a and to a dialysate outlet line 102 leading away from the dialysis liquid chamber 303a, which conducts dialysate, i.e., used dialysis fluid. Suitable connectors 145, 147 on the dialysis liquid inlet line (104) and on the dialysate outlet line 102 are used for this purpose.

Dialysis liquid chamber 303a and blood chamber 303b are separated from each other by a mostly semi-permeable membrane 303c. It represents the separating divide between the blood side with the extracorporeal blood circuit 300 and the machine side with the dialysis liquid or dialysate circuit, which is shown to the left of the membrane 303c in FIG. 1.

The arrangement of FIG. 1 further includes a valve V24, which is arranged in the dialysis inlet line 104 upstream of the first connector 145, but downstream of a first pressure sensor S03. It further includes a valve V25 which is arranged in the dialysate outlet line 102, downstream of the second connector 147, but upstream of a second pressure sensor PS4.

The arrangement of FIG. 1 includes an optional detector 315 for detecting air and/or blood. The arrangement of FIG. 1 further includes one or two pressure sensors PS1 (upstream of the blood pump 101) and PS2 (downstream of the blood pump 101, it measures the pressure upstream of the blood filter 303 (“pre-hemofilter”)) at the locations shown in FIG. 1. Other pressure sensors may be provided, e.g., pressure sensor PS3 downstream of venous blood chamber 329.

An optional single-needle chamber 317 is used in FIG. 1 as a buffer and/or compensating reservoir in a single-needle procedure, in which the patient is connected via only one of the two blood lines 301, 305 to the extracorporeal blood circuit 300.

The arrangement in FIG. 1 includes additionally an optional detector 319 for detecting air bubbles and/or blood.

An addition point 325 for Heparin may optionally be provided.

On the left of FIG. 1, a mixing device 163 is shown which provides a predetermined mixture for the respective solution from containers A (for A concentrate via concentrate supply 166) and B (for B concentrate via concentrate supply 168) for use by the treatment apparatus 2000. The solution includes water from the water source 155, (on-line, e.g., as reverse osmosis water or from bags) heated, for example, in the heating device 162.

A pump 171, which can be referred to as a concentrate pump or a sodium pump, is fluidically connected to the mixing device 163 and a source of sodium, such as the container A, and/or conveys from it. An optional pump 173, which is assigned to the container B, for example for bicarbonate, can be seen.

The herein referred to optional compressor is not shown in FIG. 1.

Furthermore, an outlet 153 for the effluent can be seen in FIG. 1. An optional heat exchanger 157 and a first flow pump 159 suitable for degassing complete the arrangement shown.

The pressure sensor PS4 downstream of the blood filter 303 on the water side, but preferably upstream of the ultrafiltration pump 131 in the dialysate outlet line 102 may be provided for measuring the pressure in the dialysate outlet line 102, for example, for measuring the filtrate pressure or membrane pressure of the blood filter 303.

Blood which exits the blood filter 303 flows through an optional venous blood chamber 329, which may include a de-aeration device 318 and be in fluid communication with the pressure sensor PS3.

The exemplary arrangement shown in FIG. 1 includes the control device or closed-loop control device 150. It may be in a wired or wireless signal connection with any of the components mentioned herein—especially or in particular with the blood pump 101—to control or regulate the treatment apparatus 2000

By using the device for on-line mixing of the dialysis liquid, a variation of its sodium content, controlled by the control device 150, is possible within certain limits. For this purpose, in particular the measured values determined by the conductivity sensors 163a, 163b may be taken into account. Should an adjustment of the sodium content of the dialysis liquid (sodium concentration) or of the substituate turn out to be necessary or desired, this can be done by adjusting the conveyance rate of the sodium pump 171.

In addition, the treatment apparatus 100 includes means for conveying fresh dialysis liquid and dialysate. A first valve may be provided between the first flow pump 159 and the blood filter 303, which first valve opens or closes the inflow towards the blood filter 303 at the inlet side. A second, optional pump or flow pump 169 which conveys dialysate towards the drainage line 153 is provided, e.g., downstream of the blood filter 303. A second valve may be provided between the blood filter 303 and the second flow pump 169, which second valve opens or closes the outflow at the outlet side.

Furthermore, the treatment apparatus 2000 optionally includes a device 161 for balancing the flow flowing into and out of the dialyzer 303 on the machine side. The device 161 for balancing is preferably arranged in a line section between the first flow pump 159 and the second flow pump 169.

The treatment apparatus 2000 further includes means, such as the ultrafiltration pump 131, for the precise removal of a volume of liquid from the balanced circuit, as predetermined by the user and/or by the control device 150.

Sensors such as the optional conductivity sensors 163a, 163b serve to determine the conductivity, which in some embodiments is temperature-compensated, as well as the fluid flow upstream and downstream of the blood filter 303.

Temperature sensors 165a, 165b may be provided as one or a plurality thereof. Temperature values supplied by them may according to the present invention be used to determine a temperature-compensated conductivity.

Further flow pumps in addition or alternatively to, e.g., the one with the reference numeral 169 may also be provided.

A number of optional valves are each denoted with V in FIG. 1; by-pass valves with VB.

In some embodiments, the control device 150 may determine the electrolyte balance and/or liquid balance based on the measured values from the aforementioned optional sensors.

Filters F1 and F2 can be connected in series.

Even when using non-pure water, the filter F1 exemplarily serves herein to generate sufficiently pure dialysis liquid by the mixing device 163, which then flows through the blood filter 303, e.g., using the counter-current principle.

The filter F2 exemplarily serves herein to generate sterile or sufficiently filtered substituate from the sufficiently pure dialysis liquid leaving the first filter F1, by filtering, e.g., pyrogenic substances. This substituate may then be safely added to the extracorporeally flowing blood of the patient and thus ultimately to the patient's body.

The treatment apparatus 2000 is optionally shown in FIG. 1 as a device for hemo(dia)filtration. However, hemodialysis apparatuses are also covered by the present disclosure, although not specifically represented in a figure.

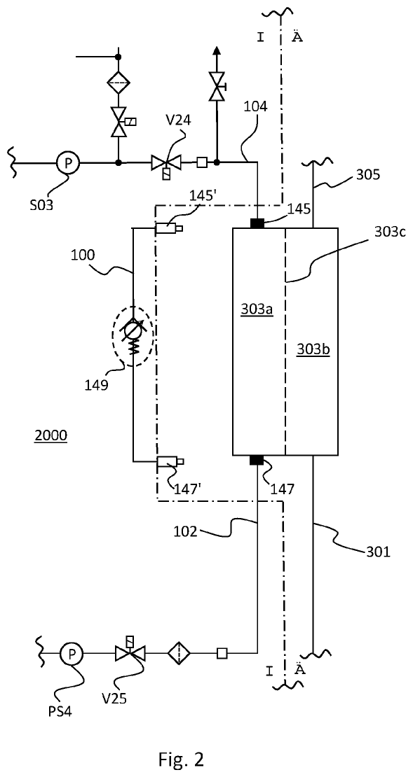

A possible position of the bypass line 100 within the blood treatment apparatus 2000 can be seen. It is described in more detail with regard to FIG. 2.

The present disclosure is not limited to the embodiment described above; this serves for illustrative purposes only.

The arrows shown in FIG. 1 generally indicate the direction of the flow in FIG. 1.

FIG. 2 shows, schematically simplified, a section of the fluid line diagram of FIG. 1 with a bypass line 100. Reference is therefore made to the description of FIG. 1.

The bypass line 100 is designed or provided to be connected, after the end of a treatment session, with a first end via its connector 145′ to the first connector 145 of the dialysis liquid inlet line 104 of the treatment apparatus 2000 and with a second end via connector 147′ to the second connector of the dialysate outlet line 102 with the aim of establishing fluid communication with the respective lines 102, 104 or short-circuiting them.

Using the dialysis liquid inlet line 104 of the treatment apparatus 2000, dialysis liquid is fed out of the interior I of the treatment apparatus 2000 and into an exterior A of the treatment device 2000 as intended, or dialysis liquid is supplied from the treatment apparatus 2000 to the blood filter 303.

Using the dialysate outlet line 102 of the treatment apparatus 2000, dialysate is returned from the exterior A to the interior I of the treatment apparatus 2000 as intended, or dialysate is returned from the blood filter 303 to the treatment apparatus 2000.

In FIG. 2, interior I and exterior A are indicated separately using a dash dot line.

The bypass line 100 furthermore has at least one non-return valve 149, here, purely by way of example, a check-valve, which allows the flow through the bypass line 100 only, or essentially only, from its first connector 145′ towards its second connector 147′ or hinders or prevents a flow in the opposite direction. In this way, after the treatment and after the intended connection of both the dialysis liquid inlet line 104 to the bypass line 100 and the dialysate outlet line 102 to the bypass line 100, a flow of fluid along and in the bypass line 100 from the dialysate outlet line 102 to the dialysis liquid inlet line 104 can be prevented. During the intended use, a flow starting from the dialysate outlet line 102 towards the dialysis liquid inlet line 104, or a fluidic connection starting from the connector of the dialysate outlet line 147 towards the connector of the dialysis liquid inlet line 145, but not in the reverse direction, is prevented.

If the connectors 145′ and 147′ are interchanged when connecting, that is, if the first connector 145′ of the bypass line 100 is accidentally connected to the connector of the dialysate outlet line 147 and consequently the second connector 147′ of the bypass line 100 is connected to the connector of the dialysis liquid inlet line 145, which corresponds to a condition of the bypass line 100, a fluid flow within the bypass line 100 from the second connector 147′ to the first connector 145′ would be prevented by the non-return valve.

Since with such a faulty connection, for example an incorrect insertion of the connectors, i.e., the connectors are interchanged when connecting the two lines 102, 104 to the bypass line 100, no fluid flow is possible over the bypass line 100 or along it, a pressure increase occurs in the dialysis liquid inlet line 104, if, for example, fluid is delivered along the dialysis liquid inlet line 104 in the direction of the bypass line 100, with the first flow pump 159 as a conveying device. This pressure increase can be detected, for example, via the pressure measured by the pressure sensor S03. For example, the change in pressure between successive points in time, for example the first and the second point in time, can be determined and used as a pressure value. If the pressure value indicating the change in pressure exceeds a first reference value, a faulty connection status can be concluded. A corresponding signal can be issued.

In an alternative embodiment, fluid is conveyed via the second flow pump 169 and/or the ultrafiltration pump 131 and the pressure in the dialysate outlet line 102 is checked or monitored. If there is a sufficiently large pressure drop in the dialysate outlet line 102, for example measured at a reference value, it can again be concluded that there is a faulty connection, since the non-return valve should open if the bypass line 100 is correctly connected to the connectors 145 and 147, but which it is noticeably not doing in view of the pressure drop. A corresponding signal that indicates “faulty connection” as a status can be issued.

Likewise, the “faulty connection” status can be recognized by comparing the pressure prevailing in the dialysis liquid inlet line 104 with the pressure prevailing in the dialysate outlet line 102. If the pressure difference (as an example of a pressure value) between the pressure measured in the dialysis liquid inlet line 104 and the pressure measured in the dialysate outlet line 102 exceeds a predetermined reference value, such as a second reference value, a faulty connection can also be concluded.

By evaluating pressure values based on pressures such as those measured by means of pressure sensor S03 or pressure sensors S03 as well as PS4, and by comparing them with one or more reference values, e.g., the first or second reference value, a faulty connection can be detected as a status and stored and/or displayed to a user.

For example, after a faulty connection has been detected, a disinfection program can be carried out in the blood treatment apparatus 2000. This can be initiated by the control device 150, which can be programmed to automatically initiate such disinfection.

Additionally or alternatively, as a status of the bypass line 100 or the non-return valve 149 it can be detected as to whether or if it—possibly despite a correct connection, see above—does not work correctly or not as intended, but instead requires, for example, higher pressures to open it than intended by the manufacturer, which also describes a status. A method described below can be carried out to check for such a status.

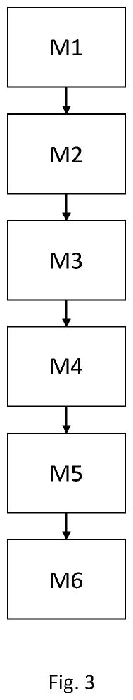

FIG. 3 shows schematically simplified, the course of the method described herein in an exemplary embodiment. The reference signs given refer to the descriptions of FIGS. 1 and 2.

In this embodiment, the method includes as M1 measuring or otherwise determining at a first time point a pressure prevailing within the dialysis liquid inlet line 104 and/or within the dialysate outlet line 102, also referred to herein as the initial pressure.

In this, it can be determined, for example, that before M2 the same or essentially the same pressures are present at the first pressure sensor S03 and at the second pressure sensor PS4.

M1 is optional.

M2 represents the step of actuating at least one conveying device, which here is exemplarily arranged for conveying fluid within the dialysate outlet line 102, with the aim of building up pressure or negative pressure against the non-return valve of the bypass line 100. Actuating the at least one conveying device preferably takes place with an open first valve V24, which is arranged here exemplarily upstream of the first connector 145, but preferably downstream of the first pressure sensor S03, and preferably with an open or closed second valve V25, which is arranged, for example, downstream of the second connector 147, but preferably upstream of the second pressure sensor PS4.

The at least one conveying device may be the ultrafiltration pump 131 and/or a second flow pump or charge pump 169.

Actuating the at least one conveying device may encompass building up a negative pressure or pressure upstream of the first connector 145 as well as downstream of the second connector 147. Hereby, a negative pressure between 150 hPa and 400 hPa (−150 hPa to −400 hPa), preferably a negative pressure between 250 hPa and 350 hPa (−(−250 hPa to −350 hPa, more preferably a negative pressure of 300 hPa (−300 hPa) may be set. At the time point at which this negative pressure is or is being set, the dialysis liquid inlet line 104 as well as the dialysate outlet line 102 are connected to the bypass line 100. By operating the conveying device downstream of the pressure sensor PS4, a negative pressure can thus be set in at least part of the dialysis liquid inlet line 104, the bypass line 100 and/or the dialysate outlet line 102.

The method includes as M3 measuring or determining a pressure prevailing within the dialysis liquid inlet line 104 and/or within the dialysate outlet line 102, at a second time point which is after the first time point.

After the second time point, a connection to the atmosphere can be established downstream of the second connector 147 and/or to the dialysate outlet line 102.

After the negative pressure in the dialysate outlet line 102 has been released, the pressure in the dialysis liquid inlet line 104 and in the dialysate outlet line 102 is measured again at a third time point.

The release of the negative pressure in the dialysate outlet line 102 can take place by opening the dialysate outlet line 102 to the environment. In this case, the release can be achieved by opening the dialysate outlet line 102 to the drain 153 by opening a connection to a drain or outlet line. Alternatively, the release of the negative pressure may be achieved by opening the dialysate outlet line 102 to a fresh water path. In this case, the dialysate outlet line 102 may be connected to a fresh water path after or downstream of the water source 155 and before or upstream of the concentrate feed via a connection point not shown here.

During the adjustment as well as the release of the negative pressure in the dialysate outlet line 102, preferably no refilling of the dialysis liquid inlet line 104 with dialysate takes place. This can be achieved, for example, by stopping the balancing device 161.

The measuring, at the first time point and/or at the second time point and/or at the third time point, of the pressure prevailing inside the dialysis liquid inlet line 104 and/or inside the dialysate outlet line 102 can be carried out using a first pressure sensor S03 arranged upstream of the first connector 145 and/or using a second pressure sensor PS4 arranged downstream of the second connector 147.

M4 represents determining a change in the pressure within the dialysis liquid inlet line 104 and/or within the dialysate outlet line 102 based on the measured values obtained at the first time point and/or the measured values obtained at the second time point and/or the measured values obtained at the third time point by measuring the prevailing pressure. The detected change in pressure is included in the following as a pressure value.

M5 corresponds to evaluating the detected change with predetermined conditions. Such conditions can be or include, for example, limit values, threshold values, reference values or ranges, value ranges for the detected change, but also, for example, the duration until a predetermined change occurs is detected, etc.

If it is determined at the third time point, i.e., after the negative pressure in the dialysate outlet line 102 has been released, that not only the negative pressure in the dialysate outlet line 102 but also the negative pressure in the dialysis liquid inlet line 104 has been released, it can be concluded that the bypass line 100 is in a faulty condition or is malfunctioning. A faulty condition of the bypass line 100 can be a state caused by a defect in the non-return valve 149 of the bypass line 100, which is in itself correctly connected to the dialysis liquid inlet line 104 and the dialysate outlet line 102, or by the non-existence of the non-return valve 149 (e.g., when using a bypass line without non-return valve).

The evaluation can be carried out by detecting the pressure in the dialysis liquid inlet line 104 and/or in the dialysate outlet line 102 after the negative pressure has been released. If the non-return valve 149 is defective or not present, a backflow can occur from the dialysate outlet line 102 in the direction of the dialysis liquid inlet line 104. Consequently, not only the pressure in the dialysate outlet line 102 increases, but also, and possibly to the same degree or extent, in the dialysis liquid inlet line 104.

As described above, pressure can be recorded at three points in time. Alternatively, the evaluation can also be based on a comparison of the pressure as a pressure value in the dialysis liquid inlet line 104 at the second time, i.e., after the build-up of the negative pressure in the dialysate outlet line 102, and at the third time point, i.e., after the release of the negative pressure in the dialysate outlet line 102. If a comparison of the pressures present in the dialysis liquid inlet line 104 at these times, e.g., with each other or with a preceding pressure, shows, for example, that a limit value has been exceeded, e.g., an increase in the pressure in the dialysis liquid inlet line 104, it can be concluded that fluid is flowing back from the dialysate outlet line 102 into the dialysis liquid inlet line 104. A faulty condition of the bypass line 100 is therefore present.

Thus, in this alternative embodiment, it is sufficient detecting the pressure only in the dialysis liquid inlet line 104 and/or preferably only at a time point after setting a negative pressure in the dialysate outlet line 102 and at a time after releasing the negative pressure in the dialysate outlet line 102.

In a further alternative embodiment, the evaluation can be based on a comparison of the pressure in the dialysis liquid inlet line 104 after the release of the negative pressure (after the third time point) with a pressure in the dialysate outlet line 102. If the pressure difference between these pressures as a pressure value is, for example, below a limit value, it can be concluded that the bypass line 100 is in a faulty condition. Since no backflow takes place when the non-return valve 149a is in fault-free condition, the negative pressure in the dialysis liquid inlet line 104 is maintained, while the negative pressure in the dialysate outlet line 102 is released, whereby a pressure difference between the dialysis liquid inlet line 104 and dialysate outlet line 102 is set or is established.

In a further alternative embodiment, after the third point in time, that is, after the negative pressure has been released in the dialysate outlet line 102, the latter is shut off downstream so that there is no fluid communication to the drain 153 or to the fresh water source 155. The pressure change is then determined as a pressure value in the dialysate outlet line 102 using at least two pressure measurements. If there is a drop in the pressure in the dialysate outlet line 102, a faulty condition of the bypass line 100 can also be inferred, as there is a backflow via the non-return valve 149 into the dialysis liquid inlet line 104, in which there is still negative pressure. A corresponding signal can be issued in each case.

Finally, M6 represents outputting a corresponding signal, for example a message or an alarm, to the user of the treatment apparatus 2000 as to whether or not the change satisfies the predetermined conditions, i.e., whether or not a faulty condition of the bypass line 100 was determined. In some embodiments, this signal can also include indications to the user, for example that the hydraulic system needs to be disinfected before it is used again.

In some embodiments, this signal can be fed to the control device and via the latter triggered by it, can lead to a disinfection, blocking the use of the treatment apparatus or some operating modes before the next treatment, before the next cleaning or disinfection or the like, etc.

### LIST OF REFERENCE NUMERALS

100 short-circuit or bypass line
2000 medical treatment apparatus
2001 housing
101 blood pump
102 dialysate outlet line
104 dialysis liquid inlet line
105 substituate line
107 pre-dilution valve
107a line belonging to the pre-dilution valve
109 post-dilution valve
109a line belonging to the post-dilution valve
111 substituate pump
131 ultrafiltration pump
145 connector of the dialysis liquid inlet line
145′ first connector of the bypass line
147 connector of the dialysate outlet line
147′ second connector of the bypass line
149 non-return valve; check valve
150 control device
153 drain
155 water source
157 heat exchanger
159 first flow pump
161 balancing device
162 heating device
163 mixing device
163a conductivity sensor
163b conductivity sensor
165a temperature sensor
165b temperature sensor
166 concentrate supply
168 concentrate supply
169 second flow pump
171 pump, sodium pump
173 pump, bicarbonate pump
300 extracorporeal blood circuit
301 first line (arterial line section)
302 (first) tubing clamp
303 blood filter or dialyzer
303a dialysis liquid chamber
303b blood chamber
303c semi-permeable membrane
305 second line (venous line section)
306 (second) tubing clamp
315 detector
317 single-needle chamber
318 de-aeration device
319 detector
325 addition point for Heparin
329 venous blood chamber
F1 filter
F2 filter
A container
B container
Ä exterior
I interior
M1 to M6 method steps
P pressure measurement points
PS1 arterial pressure sensor (optional)
PS2 arterial pressure sensor (optional)
PS3 pressure sensor (optional)
PS4 second pressure sensor for measuring filtrate pressure (optional)
S03 first pressure sensor
V valves
V24 valve in dialysis liquid inlet line
V25 valve in dialysate outlet line
VB bypass valve
Y Y-connector

1-15. (canceled)

16. A bypass line for establishing fluid communication between: (i) a dialysis liquid inlet line of a medical treatment apparatus by which dialysis fluid is fed out of an interior of the treatment apparatus into an exterior of the treatment apparatus, and (ii) a dialysate outlet line by which dialysate is fed back from the exterior into the interior of the treatment apparatus, the bypass line comprising:
a first connector for connecting the bypass line to achieve fluid communication with a connector of the dialysis inlet line;
a second connector for connecting the bypass line to achieve fluid communication with a connector of the dialysate outlet line; and
a non-return valve which only allows flow through the bypass line from the first connector towards the second connector.

17. A medical treatment apparatus comprising a housing and a bypass line according to claim 16.

18. The medical treatment apparatus according to claim 17, wherein the bypass line is completely or at least partially accommodated in the housing of the treatment apparatus, wherein the first connector and the second connector of the bypass line are accessible from the exterior of the treatment apparatus.

19. A medical treatment apparatus according to claim 17, further comprising the dialysis liquid inlet line having a connector provided for connection to a blood filter, the dialysate outlet line having a connector provided for connection to a blood filter, and the conveying device for conveying fluid within the dialysis liquid inlet line and/or within the dialysate outlet line.

20. A method of checking a condition of the bypass line according to claim 16 connected to a medical treatment apparatus provided with the dialysis liquid inlet line and the dialysate outlet line, wherein the treatment apparatus comprises:
the dialysis liquid inlet line with a connector provided for connection to a blood filter;
the dialysate outlet line with a connector provided for connection to the blood filter; and
a conveying device for conveying fluid within the dialysis liquid inlet line and/or within the dialysate outlet line;
wherein the method encompasses:
actuating the conveying device to deliver fluid within the dialysis liquid inlet line and/or within the dialysate outlet line to establish positive pressure or negative pressure therein;
measuring a pressure prevailing in the dialysis inlet line and/or in the dialysate outlet line at a first time point;
determining at least one pressure value based on the measured pressure;
evaluating the at least one pressure value; and
emitting or outputting a signal indicating the condition of the bypass line as a result of the evaluation.

21. The method according to claim 20, whereby the result is an indication of a faulty connection of the bypass line with the dialysis liquid inlet line and the dialysate outlet line, and/or wherein evaluating of the at least one pressure value comprises a comparison of the at least one pressure value with a first reference value or reference range.

22. The method according to claim 20, wherein the result is a faulty function of the non-return valve of the bypass line or an indication of the faulty function of the non-return valve of the bypass line; and/or the result that the faulty connection exists, is or presupposes a connection of the connector of the dialysis liquid inlet line to the second connector of the bypass line and a connection of the connector of the dialysate outlet line to the first connector of the bypass line.

23. The method according to claim 20, wherein, to determine the pressure value, both the pressure prevailing in the dialysis liquid inlet line at the first time point, and the pressure prevailing in the dialysate outlet line are or have been measured, wherein, a pressure difference is determined between the pressure measured in the dialysis liquid inlet line and the pressure measured in the dialysate outlet line or vice versa;
wherein evaluating the at least one pressure value comprises a comparison of this pressure difference with a second reference value or reference range therefor.

24. The method according to claim 20, wherein the method further comprises measuring the pressure prevailing in the dialysis liquid inlet line and/or in the dialysate outlet line at a second time point and determining at least a second pressure value based thereon;
wherein evaluating the pressure value comprises a comparison of the pressure difference between the pressure values determined in the dialysis liquid inlet line at the first and second time points and/or a comparison of the pressure difference between the pressure values determined in the dialysate outlet line at the first and second time points with a third or fourth reference value or range therefor, respectively.

25. The method according to claim 20, wherein actuating the conveying device comprises building a negative pressure in the dialysis liquid inlet line as well as in the dialysate outlet line; and wherein the method comprises releasing the negative pressure in the dialysate outlet line; wherein measuring a pressure prevailing in the dialysis liquid inlet line and/or in the dialysate outlet line comprises a measurement at one or more times after the pressure release.

26. The method according to claim 20, wherein actuating the at least one conveying device takes place when a first valve is open which is upstream of the connector of the dialysis liquid inlet line but downstream of the first pressure sensor and/or when a second valve is open, which is arranged downstream of the second connector of the dialysate outlet line but upstream of the second pressure sensor.

27. The method according to claim 26, wherein releasing the negative pressure in the dialysate outlet line is achieved by connecting the dialysate outlet line to a fresh water source or to a drain.

28. The method according to claim 27, further comprising:
fluidically shutting off the dialysate outlet line after the negative pressure in the dialysate outlet line has been released in such a way that there is no longer any connection between the dialysate outlet line and the fresh water source or the drain,
wherein, to determine the at least one pressure value, at least two pressures are measured in the dialysate outlet line at two different times points after the dialysate outlet line has been blocked, and
wherein a signal is emitted or output which is associated with a faulty condition of the bypass line when as a result of evaluating the at least one pressure value pressure change within the dialysate outlet line is detected.

29. The method according to claim 27, wherein releasing the negative pressure in the dialysate outlet line is achieved by opening a valve to a fresh water line or a valve to a drain line.

30. The method according to claim 27, wherein a signal associated with a faulty condition of the bypass line is emitted or output when:
evaluating the pressure value results in the pressure value exceeding a reference value or leaving a reference range, and/or
determining the pressure value based on pressures within the dialysis liquid inlet line measured after the pressure was released results in a pressure increase.

31. A control device programmed to carry out a test to check a condition of a bypass line connected to the dialysis liquid inlet line and the dialysate outlet line of a medical blood treatment apparatus, the test comprising the method according to claim 20.

32. A medical treatment apparatus comprising the control device according to claim 31.
